(19) United States
(12) Patent Application Publication
(10) Pub. No.: US 2016/0206200 A1
(43) Pub. Date: Jul. 21, 2016

(54) DEVICE AND METHOD FOR MONITORING INTERNAL ORGANS

(71) Applicants: Michael POSCENTE, Calgary (CA); Orly YADID-PECHT, Calgary (CA); Christopher ANDREWS, Calgary (CA); Martin MINTCHEV, Calgary (CA); UTI LIMITED PARTNERSHIP, Calgary (CA)

(72) Inventors: Michael D. Poscente, Calgary (CA); Orly Yadid-Pecht, Calgary (CA); Christopher N. Andrews, Calgary (CA); Martin P. Mintchev, Calgary (CA)

(73) Assignee: UTI Limited Partnership, Calgary, AB (CA)

(21) Appl. No.: 14/914,397

(22) PCT Filed: Aug. 22, 2014
(86) PCT No.: PCT/IB14/64027

§ 371 Date: Feb. 25, 2016

Related U.S. Application Data
(60) Provisional application No. 61/959,670, filed on Aug. 30, 2013.

(51) Int. Cl. A61B 5/00

(52) U.S. Cl. CPC A61B 5/0031; A61B 5/0028; A61B 5/6861; A61B 5/6852

(57) ABSTRACT

The present invention provides a method and an apparatus for minimally-invasive diagnosis of conditions within the body of an animal. In one particular embodiment, the present invention provides a method and an apparatus for utilizing intraluminally-generated signals to diagnose disorders or monitor the function of internal organ of an animal by assessing the signal transcutaneously. The signals can be electrical, magnetic, electromagnetic, acoustic, ultrasonic, optical, etc.

### CROSS-REFERENCE TO RELATED APPLICATIONS

This application claims the priority benefit of U.S. Provisional Application No. 61/959,670 filed Aug. 30, 2013, which is incorporated herein by reference in its entirety.

### FIELD OF THE INVENTION

The present invention relates generally to the minimally-invasive diagnosis of conditions within the body of an animal. In one particular embodiment, the present invention relates to generating a signal within the interior of the animal's body and assessing the signal transcutaneously to diagnose monitor or diagnose an internal organ of the animal. The signal can be electrical, magnetic, electromagnetic, acoustic, ultrasonic, optical, etc.

### BACKGROUND OF THE INVENTION

Impedance-based measurements of internal organs have been suggested in the prior art, but as a rule, they are not transluminal and transverse to a particular organ or interest, and because of that, they cannot present a reliable dynamic picture of the functional characteristics of the organ. The aim of the present invention is to fill this void by presenting a comprehensive method and devices to provide dynamic, real-time transverse impedance measurements from internal organs. As examples of such organs, the stomach and the liver are considered. As an example of wave signal, electromagnetic waves are considered.

Gastric motility refers to the mechanism by which gastric contents are propelled into the duodenum. This can be compromised by several disorders, the two most prevalent of which are functional dyspepsia and gastroparesis.

Functional dyspepsia affects 20-40% of the population, and is associated with pain or discomfort in the upper abdomen without obvious organic cause. It is poorly understood because or its vague diagnostic criteria. About 30% of patients diagnosed with functional dyspepsia also suffer from delayed gastric emptying, one of the key features of gastroparesis.

Gastroparesis is a chronic disorder characterized by delayed gastric emptying with no physical obstruction. It is associated with early satiety, nausea, vomiting, bloating, upper abdominal pain, and in diabetic cases can have a drastic impact on blood glucose management.

Several techniques exist to diagnose delayed gastric emptying, including gastric emptying scintigraphy and breath tests, both of which are suggested in U.S. Pat. No. 8,317,718 (Nov. 27, 2012) by Bush et al., Mill, ultrasonography, pressure manometry as described in U.S. Pat. No. 5,609,060 (Mar. 11, 1997) by Dent, and cutaneous electrogastrography as described by U.S. Pat. No. 6,249,697 (Jun. 19, 2001) by Asano. Scintigraphy is presently the most accurate and commonly used test, however it is expensive, time consuming, involves radiation, and is not fit for ambulatory testing. It is particularly limited and affected by body position. Many other tests are limited by cost or low accuracy, and do not offer a means to utilize transluminal transverse impedance measurements to assess gastric function.

Electrical impedance measurements of liver steatosis have been proposed in U.S. Patent Application 2010/0100002(Apr. 22, 2010) by Villa Sanz et al. They utilized a non-transverse 4-electrode configuration and electrical frequencies in the range of 1 to 100 kHz to assess liver steatosis. However, this technology does not offer means to utilize transluminal, transverse impedance measurements to assess liver stiffness.

A technique for monitoring liver stiffness has been proposed in U.S. Pat. No. 8,118,744 (Feb. 21, 2012). They utilize a non-transverse shear wave generator and receiver to assess liver stiffness, however the setups can be challenged in their ability to couple enough energy through the skin and subcutaneous fat to reach organs such as the liver, especially in obese patients. This technology does not offer a means to utilize transluminal, transverse impedance measurements to assess liver stiffness.

Despite many advances, there is a continuing need for minimally invasive technology to monitor internal organs.

### SUMMARY OF THE INVENTION

Some aspects of the invention provide a device and a method for monitoring internal organs using minimally invasive technique. In particular, the invention provides a device and a method for using transluminal electrical signal measurement to monitor internal organs. Such a device and method offer inter alia minimal invasive monitoring of internal organs thereby significantly reducing, the cost and minimizing or preventing possible discomfort and/or side-effects observed by conventional devices and methods.

A technique for using transluminal, transverse electrical signal measurements to interrogate internal organs for the purposes of diagnosing an abnormality in the interrogated organ such as, for example, gastric motility dysfunction or liver stiffness, is described herein. In some embodiments, the diagnostic technique involves recording the attenuation of a wave signal induced transluminally across the organ of interest, and uses the recorded attenuation to determine whether abnormality of the interrogated organ is present such as, for example, gastric motility dysfunction affecting the stomach or abnormal hepatic stiffness affecting the liver.

In other embodiments, the method utilizes a wave signal of specific frequency and amplitude emitted from within the lumen of a given gastrointestinal organ.

Yet in other embodiments, the method includes using a cutaneous sensor that is placed over the abdominal projection of the organ of interest to detect the signal emitted from within the lumen of the internal organ of interest. Processing of the measured signal can be used to reveal information about the interrogated organ such as, for example, if gastric motility dysfunction, or abnormal liver stiffness is present.

Still in other embodiments, the method can also determine the location, temporal, and/or spectral characteristics of the signals. In some instances, the location information is used to selectively process signals that may be indicative of dysfunction of the interrogated organ. Alternatively or additionally the technique can be used to compare the temporal and/or spectral information of the transluminally attenuated signals to the temporal and/or spectral characteristics of the transluminally attenuated signals within a normal or healthy patients. The technique can, based on comparison, provide a diagnostic output to a user.

In accordance with one particular aspect of the invention, a system or apparatus for monitoring internal organ function can optionally include a computer readable medium, and software stored on the computer readable medium and adapted to be executed by a processor. In some embodiments, when executed by the processor, the software causes the processor to acquire one or more signals associated with the transluminal attenuation by the interrogated organ, calculate a characteristic of the attenuated signal and determine whether the characteristic corresponds to an abnormality associated with the interrogated organ.

In accordance with another aspect of the invention, a method of monitoring internal organ function can include acquiring or detecting transluminal transverse impedance measurements associated with a body and can include comparing the acquired information to information associated with a healthy condition. The method can be used to identify organ abnormality based on the comparison of the acquired transluminal transverse impedance information to the information associated with a healthy condition.

One particular aspect of the invention provides a method for monitoring function of an internal organ of an animal. Such aspect of the invention comprises placing a signal emitting or generating device inside a body of an animal. Typically, the signal emitting device is placed within the lumen of an internal organ to be monitored. Alternatively, the signal emitting device is place on the surface of the organ to be monitored. Still in some embodiments, the signal emitting device is place on the lumen or the surface of an organ that is near the internal organ to be monitored. For example, to monitor function of an animal's liver, one can place the signal emitting device inside the stomach near the liver or on the surface of the stomach near the liver of the animal.

In some embodiments, the signal emitting device comprises a signal transmitter and a power source to operate the signal transmitter. Yet in other embodiments, the signal emitting device also includes a signal or a wave inducer that operates the signal transmitter. Such a signal inducer can be attached to the signal transmitter via a wire or it can be configured to control the signal transmitter remotely, for example, by blue tooth, wi-fi, infrared or electrical remote control.

The method of the invention also includes transcutaneously measuring or detecting the signal emitted by said signal emitting device. Typically, a signal detector is placed on the exterior body surface of the animal. In this manner, only the signal emitting device is placed within the body interior of the animal. Cutaneous placement of the signal detector allows non-invasive or minimally invasive monitoring of an internal organ of the animal.

In some embodiments, the method further comprises the step of converting the analog signal to a digital signal.

The method also includes analyzing the detected signal to determine the function of the internal organ. In some embodiments, said step of determining the function of the internal organ using the measured signal comprises comparing the measured signal to a control signal measurement. In some instances, said control signal measurement comprises measured signal of the animal under normal condition. In other instances, said control signal measurement comprises measured signal of a control animal whose internal organ functions normally.

Still another particular aspect of the invention comprises an apparatus for transcutaneously monitoring function of an internal organ of an animal. In this particular aspect of the invention, the apparatus includes a signal generator configured to be placed within the interior of the body of an animal. Typically, the signal generator is configured to be placed within the lumen of an internal organ to be monitored. In some embodiments, the signal generator device is configured to be place on the surface of the organ to be monitored. Still in some embodiments, the signal generator device is configured to be place on the lumen or the surface of an organ that is near the internal organ to be monitored. For example, to monitor function of an animal's liver, the signal generator device can be configured to be placed inside the stomach near the liver or on the surface of the stomach near the liver of the animal.

The apparatus of the invention also includes a signal detector configured to transcutaneously detect the signal generated by said signal generator when said signal generator is placed within the interior of the animal's body. Typically, the signal detector is configured to be placed on the exterior body surface of the animal. In this manner, only the signal generating device is placed within the interior of the animal's body. Cutaneous placement of the signal detector allows non-invasive or minimally invasive monitoring of an internal organ of the animal.

The apparatus of the invention also includes an analyzer operatively connected to said signal detector. The nanalyzer is configured to analyze the signal detected by said signal detector.

In some embodiments, the signal generator further comprises a retaining element configured to retain said signal generator within the interior of the animal's body or the lumen of an internal organ of the animal for a period of time. In some instances, said retaining element comprises a biocompatible polymeric material. Typically, said biocompatible polymeric material comprises biodegradable polymer. Yet in other embodiments, the retaining element comprises an endoscope.

Typically, said signal detector is configured to generates a distinct signal based on the level or the amount of detected signal.

In some embodiments, said signal generator generates an electrical signal. In such embodiments, the signal detector typically comprises at least one electrode.

### BRIEF DESCRIPTION OF THE DRAWINGS

FIG. 1 is a schematic block diagram of a system that uses transluminal electrical signal (e.g., transverse impedance) measurements to monitor function of an internal organ of an animal.

FIG. 2 is a diagram depicting one particular example of a catheter based internal organ monitoring apparatus of the present invention>

FIG. 3 is a schematic illustration showing one particular embodiment of the invention for using a catheter and cutaneous sensors to monitor stomach function.

FIG. 4 a schematic illustration showing one particular embodiment of the invention for using a catheter and cutaneous sensors to monitor the liver function.

FIG. 5 is a schematic diagram depicting one particular embodiment of an orally administrable electrical signal generator of the invention.

FIG. 6 is a schematic illustration depicting one particular embodiment for monitoring the stomach function using an orally administrable electrical signal generator and a plurality of cutaneous signal detectors.

FIG. 7 is a schematic diagram illustrating how transluminal transverse impedance measurement can be achieved.

FIG. 8 shows the position of the force transducers sutured to the serosa of the stomach (A) and the intraluminal position of the expanded gastric retentive pill carrying the TIIM oscillator (B).

FIG. 9 shows an oscilloscope reading from the gastric serosa prior to the force transducer implantation verified the presence of an activated TIIM pill (A). The sham pills did not have any signal (B).

FIG. 10 is a combined plot of the raw signals and the 1-minute motility indices for an active pill in the baseline state (seconds 0-1800) and after the administration of neostigmine (seconds 1800-3600). The 1-minute duration for the administration of neostigmine is denoted with a thick vertical line.

FIG. 11 is a combined plot of the raw signals and the 1-minute motility indices for an inactive pill in the baseline state (seconds 0-1800) and after the administration of neostigmine (seconds 1800-3600). The 1-minute duration for the administration of neostigmine is denoted with a thick vertical line.

### DETAILED DESCRIPTION OF THE INVENTION

The present invention will be described with regard to the accompanying drawings which assist in illustrating various features of the invention. In this regard, the present invention generally relates to a method and an apparatus for monitoring function of an internal organ of an animal using a transcutaneous measurement of electrical signal generated within the lumen of the internal organ to be monitored. That is, the invention relates to an apparatus comprising an electrical signal generator configured to be placed within a lumen of an internal organ the animal whose internal organ function is to be monitored; an electrical signal detector configured to transcutaneously detect electrical signal generated by said electrical signal generator when said electrical signal generator is placed within the lumen of the internal organ of said animal; and an analyzer operatively connected to said electrical signal detector, wherein said analyzer is configured to analyze the electrical signal detected by said electrical signal detector. The invention also relates to a method for using the same.

Various embodiments of the method for transcutaneously measuring function of an internal organ of an animal and apparatuses that can be used for such a method are generally illustrated in accompanying drawings. It should be appreciated that the accompanying drawings are provided for the purpose of illustrating the practice of the present invention and do not constitute limitations on the scope thereof.

Generally, the method and the apparatuses disclosed herein can be used to monitor internal organ function of an animal. Typically, the animal is a mammal. Often the animal is a primate, including human, canine, feline, equine, bovine, mouse, rabbit, other laboratory and domesticated animals, as well as non-domesticated animals that can be treated by a veterinarian.

The electric signal generator can be any device that is configured to be placed within a lumen of the internal organ of an animal. The electric signal generator can be configured to generate any electrical signal that can be detected and/or measured transcutaneously. For example, the electric signal generator can be used to generate oscillating (or non oscillating) impedance, voltage, current, or any other electrical signal that can be detected transcutaneously. In one particular embodiment, the electric signal generator generates oscillating electrical signal. In another embodiment, the electrical signal detector is used to transcutaneously detect and/or measure impedance.

For the sake of brevity and clarity, the invention will now be described in reference to monitoring internal organ function by measuring transcutaneous intraluminal impedance measurement (“TIIM”). However, as stated above, the scope of the invention is not limited to TIIM as any electrical signal can be used to practice the scope of the invention.

While diagnostic techniques focusing on the stomach and the liver are described herein in connection with a human patient, it should be understood that transverse transluminal impedance measurements can be applied to the esophagus, the small intestine, the spleen, the colon etc. In one particular embodiment, the function of the internal organ is monitored, for example, to determine the specific characteristics of the internal organ, or for the purposes of diagnosing abnormality or any clinical conditions associated with abnormal function of the internal organ. This can be achieved by modifying the position of the external cutaneous sensors while maintaining the scope and spirit of the present invention.

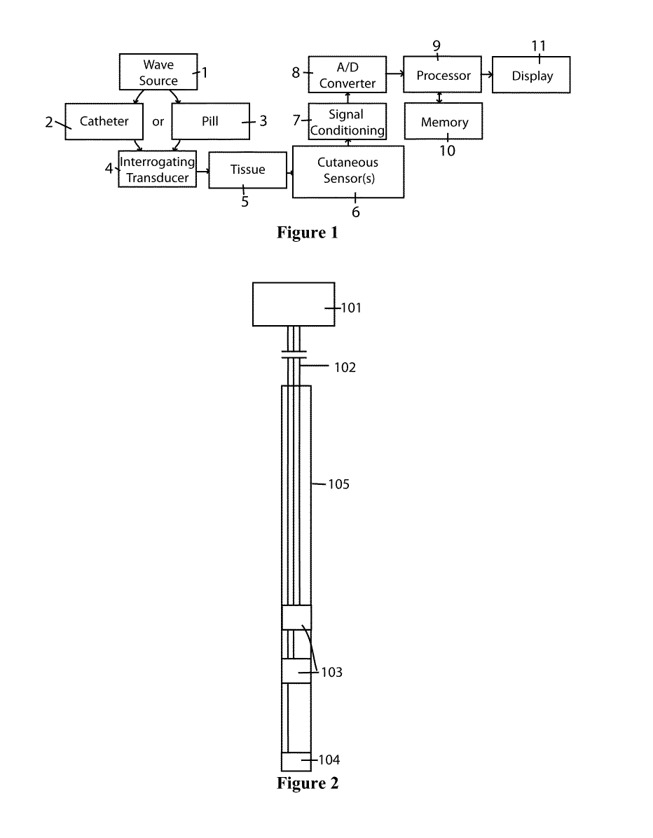

FIG. 1 is a schematic block diagram of a system that uses transluminal transverse impedance measurements to diagnose organ abnormality in a patient. As shown in FIG. 1, the system starts with a wave source 1 that is configured to send a signal to the interrogating transducer 4 (i.e., electric signal generator) held in place using either a catheter 2 or in a pill (or orally administrable) form 3. It should be appreciated that the wave source 1 can be part of the interrogating transducer 4 such that it is self-activated. Alternatively, the wave source 1 can be a separate component that is used to activate the interrogating transducer 4 after the interrogating transducer 4 is placed within the lumen of the internal organ to be monitored.

From the interrogating transducer 4 the electrical signal passes into the tissue 5 and is observed or detected transluminally using cutaneous sensors (or electrical signal detector) 6, thereby transversely measuring or determining impedance across the interrogated tissue 5. The cutaneous sensors 6 then pass the acquired or detected signal to the analyzer. The analyzer can be a simple display unit 11 that displays the detected/measured signal or in can comprises other components. For example, in FIG. 1, the analyzer includes a signal conditioning block 7. The detected electrical signal, which is often a very low power is passed through the signal conditioning block 7, which can be used to amplify the detected electrical signal and/or filter out the background noise. The electrical signal can optionally then be passed through an analog-to-digital (A/D) converter 8. The digital form of the signal can optionally be processed by a processor 9, which can be either display the signal through a compatible display unit 11 and/or stored in memory 10.

The wave source 1 can be implemented using one or more custom, semi-custom or commonly available integrated circuits. For example, if transverse electromagnetic impedance is to be assessed, a miniature oscillator TS3004 (Touchstone Semiconductor, Milpitas, Calif., USA) can be utilized. It should be reemphasized that the scope of the invention is not limited to such as discussed above. The catheter 2 can be implemented using custom, semi-custom or commonly available technology, or custom-modified available technology such as, for example a custom-modified Esophageal Z/pH catheter (Sandhill Scientific, Highlands Ranch, Colo,., USA) with built in interrogating transducers 4 or custom-added interrogating transducers 4.

The interrogating transducer 4 can be of many different forms; electrodes for inducing a small electrical signal to measure transluminal transverse electrical impedance, acoustic or ultrasonic transducers for inducing transluminal transverse acoustic or ultrasonic waves, a light emitter for inducing transluminal transverse optical impedance, etc. For example if transverse acoustic impedance is to be assessed, the interrogating transducer 4 can be an FK-23451 audio transducer (Knowles Electronics, Itasca, Ill., USA). The pill 3 can also be implemented using custom, semi-custom or commonly available technology, and can also utilize an interrogating transducer of many different forms, as described herein. Such pill can be temporarily retentive for a given gastrointestinal organ, utilizing, for example the technology described in U.S. Patent Application Publication No. 2011/0082419, filed by one of the present inventors, or non-retentive, or the type similar to the technology described in U.S. Pat. No. 8,185,211, issued to Cho et al.

The tissue 5 can be any tissue that can be or is desired to be interrogated by medical personnel to detect abnormality such as, for example, delayed gastric emptying or liver stiffness. The cutaneous sensors 6 can be contact or non-contact sensors that correlate appropriately to the interrogating transducer such as, for example cutaneous electrodes for measuring transluminal transverse electrical impedance in which case the transducer can also be an electrode, or the cutaneous sensors 6 may be microphones in the case of the interrogating transducer being acoustic in nature. In the case that the transluminal transverse impedance being measured is electrical in nature, cutaneous electrodes used for ECG (Conmed, Utica, N.Y., USA) can be used. The cutaneous sensor 6 can optionally convert the transluminal transverse impedance measurement from the attenuation of the interrogating signal by the organ of interest into a low power electrical signal which can be sent to the signal conditioning block 7 via wires or any other suitable media such as wireless RF, infrared, optical, etc. The signal conditioning block 7 optionally can include amplifiers, filters, and other circuitry to manipulate the signals from the cutaneous electrodes 6.

The signal conditioning block can include a low-pass filter, a high-pass filter, or a band-pass filter to remove undesirable noise. One example of such a signal conditioning device, which should not be considered limiting, is a bioelectric amplifier (James Long Company, Caroga Lake, N.Y., USA). The A/D converter 8 receives the conditioned signal and digitizes the analog values at a rate appropriate to avoid aliasing of the physical process. One available technology, which should not be considered limiting, is a DAQCard-A1-16XE-50 (National Instruments, Austin, Tex., USA), which can connect to a personal computer. These digital values are then processed by the processor 9. The processor 9 can be part of a personal computer, or a microcontroller integrated circuit chip, but can also be implemented using a custom integrated circuit chip, or can be implemented using any other device suitable for carrying out the methods described herein. One commonly available processor that is suitable is the ATSAMA5D31A-CU-ND (Atmel, San Jose, Calif., USA).

The memory 10 is coupled to the processor 9, and can include software that when executed by the processor 9 causes the processor to display the measurements on the display 11, or calculate temporal or spectral characteristics of the measurements. The processor 9 can be used to calculate relative attenuation of the signal measured and recorded by the cutaneous sensors 6 by having information about the interrogating signal from the wave source 1 stored in memory 10. The processor 9 can also directly store information about the interrogated organ, and the subsequent transluminal transverse impedance data from the A/D converter 8 in the memory 10 for future reference or use.

The display 11 can be any suitable display that communicates with the processor 9 and which can display graphic or textual information relating to the transluminal transverse impedance measurements from one or more cutaneous sensors 6, optionally conditioned by the signal conditioning block 7, optionally digitized by the A/D converter 8, and optionally stored in memory 10. This display 11 can facilitate medical personnel in determining if any abnormality of the interrogated organ is present. One available display technology, that should not be considered limiting, is to use the P1913S 19-inch flat panel monitor (Dell, Austin, Tex., USA).

One particular embodiment of the invention comprises a catheter-based apparatus for monitoring gastric motility. FIG. 2 is a schematic diagram depicting a catheter based embodiment of the inventive apparatus with the purpose of transluminal transverse impedance interrogation. In this particular embodiment, the wave source 101 sends a signal via wire 102 through the body of the catheter 105 to an interrogating transducer in the middle of the catheter 103 as well as or alternatively to an interrogating transducer on the tip of the catheter 104. The position and number of transducers on the body of the catheter can be modified to suit different desired measurements, while maintaining the scope and spirit of the invention. In this first embodiment, the catheter is placed in a minimally invasive fashion such as, for example orally, such that the interrogating transducer is within the lumen of an internal gastrointestinal organ such as, for example the stomach, or the small intestine. The wave source can be integrated completely, partially, or not at all into the body of the catheter in which case it would be integrated accordingly outside the body of the catheter 105.

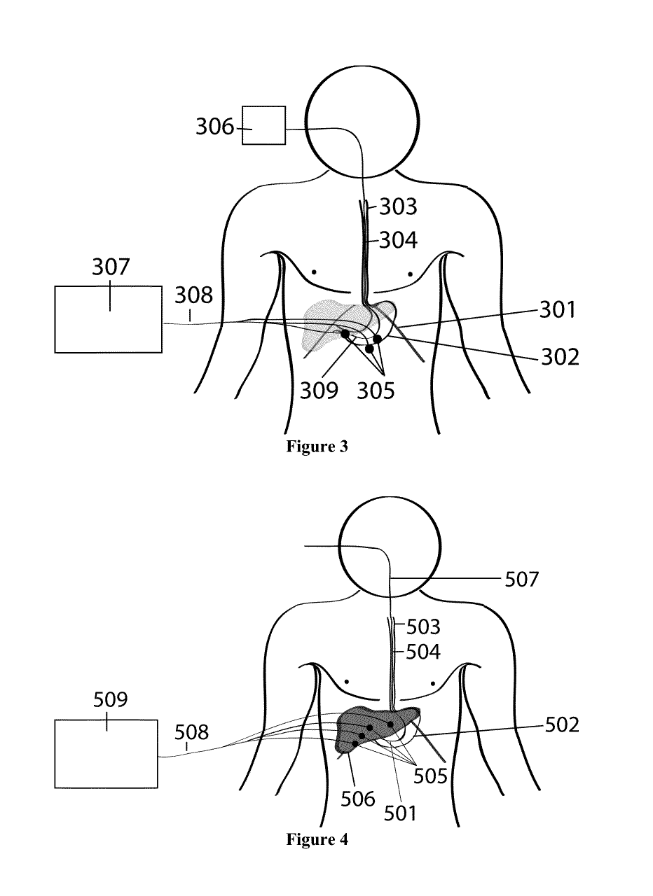

FIG. 3 is a schematic diagram illustrating the use of one particular embodiment of this inventive apparatus in which a catheter 304 is used to position the interrogating transducer 309 into the lumen of a gastrointestinal organ, in this case the stomach 302, orally via the esophagus 303. In this diagram the wave source 306 is not integrated into the body of the catheter 304. The interrogating transducer 309 transforms the wave source into a signal, which is picked up by cutaneous sensors 305 as described above. In this embodiment the cutaneous sensors 305 are positioned to measure transluminal transverse impedance of the stomach, utilizing landmarks of the body such as, for example the rib cage 301. The position and number of the cutaneous sensors 305 can be changed to measure different desired characteristics, while maintaining the scope and spirit of the present invention. The cutaneous sensors 305 convert measured phenomena from the interrogating transducer into low power electrical signals which can be transmitted via wires 308 or any other appropriate medium as described above to the final stage 307 which encompasses the signal conditioning, A/D conversion, processing, memory, and display stages shown and described in FIG. 1. While FIG. 3 depicts the signal generator as comprising a two separate components, e.g., the wave source 306 and the interrogating transducer 309, it should be appreciated that these two components can be integrated into a single device. As described above, the cutaneous sensor 305 detects the signal generated by the signal generator and the detected signal is then analyzed to determine gastric motility or any other desired function of the stomach or small intestine.

Another embodiment of the invention illustrates a catheter-based apparatus for monitoring liver function. A catheter based apparatus shown in FIG. 2 can be used for transluminal transverse impedance interrogation of the liver. This embodiment also demonstrates that simply by moving the cutaneous sensors, one can utilize the apparatus of the invention to measure the transluminal transverse impedance of different internal organs of interest.

FIG. 4 is a schematic illustration depicting a method for using the apparatus described herein to monitor the function of (or interrogate) the liver 506. In FIG. 4, the wave source 507 has been completely integrated into the catheter body 504, however the wave source 507 could also be integrated partially, or not at all as shown in FIG. 3. The interrogating transducer 501 is positioned in a minimally invasive fashion into the lumen of a gastrointestinal organ near the organ of interest. In this particular embodiment the catheter 504 is positioned via the esophagus 503 into the stomach 502, which is close to the liver 506. Since the wave source 507 emits a signal via the interrogating transducer 501 from within the gastrointestinal organ that it is positioned, the signal passes through the lumen of the gastrointestinal organ and any other internal organ in the immediate vicinity, and thus the transluminal transverse impedance dynamics of a particular organ of interest can be measured by positioning the cutaneous sensors 505 along the abdominal projection of the particular organ of interest. The position and number of cutaneous sensors 505 can be modified to measure desired characteristics from different internal organs, while maintaining the scope and spirit of the present invention. The cutaneous sensors 505 may convert measured phenomena or signal from the interrogating transducer into low power electrical signals which can be transmitted via wire 508 or any other appropriate medium as described above to the final stage 509 which optionally encompasses the signal conditioning, A/D conversion, processing, memory, and display stages as shown and described in FIG. 1.

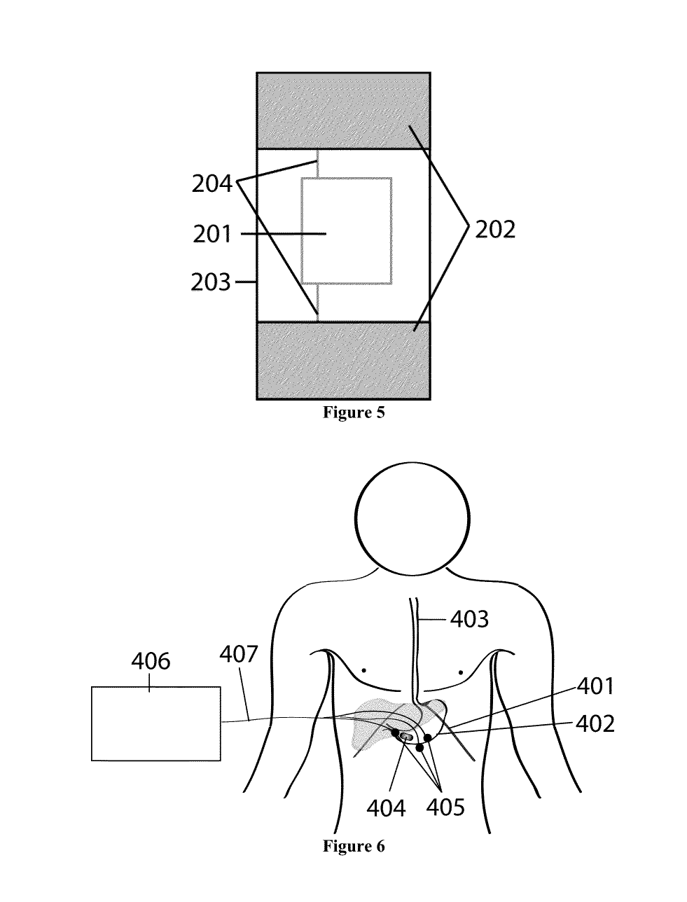

Still another embodiment of the invention provides an autonomous (e.g., capsule-based) apparatus for monitoring gastric motility. This apparatus can also be used for transluminal transverse impedance interrogation. In contrast to the catheter-based apparatus as depicted in FIG. 1, the autonomous apparatus allows placement of the signal generator within the lumen of an internal organ without the need for a catheter. FIG. 5 is a schematic illustration of one particular embodiment of the inventive apparatus in capsule form. The wave source 201 is completely contained inside the body of the capsule 203, and is connected via wires 204 to one or more interrogating transducers 202 affixed to the body of the capsule 203. Thus, the signal generator is provided as a capsule that can be orally administrable to the animal.

FIG. 6 is a schematic illustration showing use of this particular embodiment of the inventive apparatus to interrogate the stomach. The capsule body 404 encompasses the wave source 201, and interrogating transducer(s) 202 as shown in FIG. 5. The capsule 404 is positioned in a minimally invasive fashion into the stomach 402 orally via the esophagus 403. As in the previous embodiment the position of the interrogating transducer can be within the lumen of any gastrointestinal organ, provided it offers desired proximity to another internal organ. Cutaneous sensors 405 appropriately coupled to the interrogating transducer 202 and may be positioned based on landmarks on the body such as, for example the rib cage 401. As in previous embodiments the position and number of cutaneous sensors 405 can be changed to measure different characteristics from different internal organs while maintaining the scope and spirit of the invention. The cutaneous sensors 405 convert physical phenomena (e.g., signal that is detected) from the interrogating transducer into low power electrical signals that can be transmitted via wire 407 or any other appropriate medium described above to the final stage 406 which optionally encompasses the signal conditioning, A/D conversion, processing, memory, and/or display stages shown and described in FIG. 1.

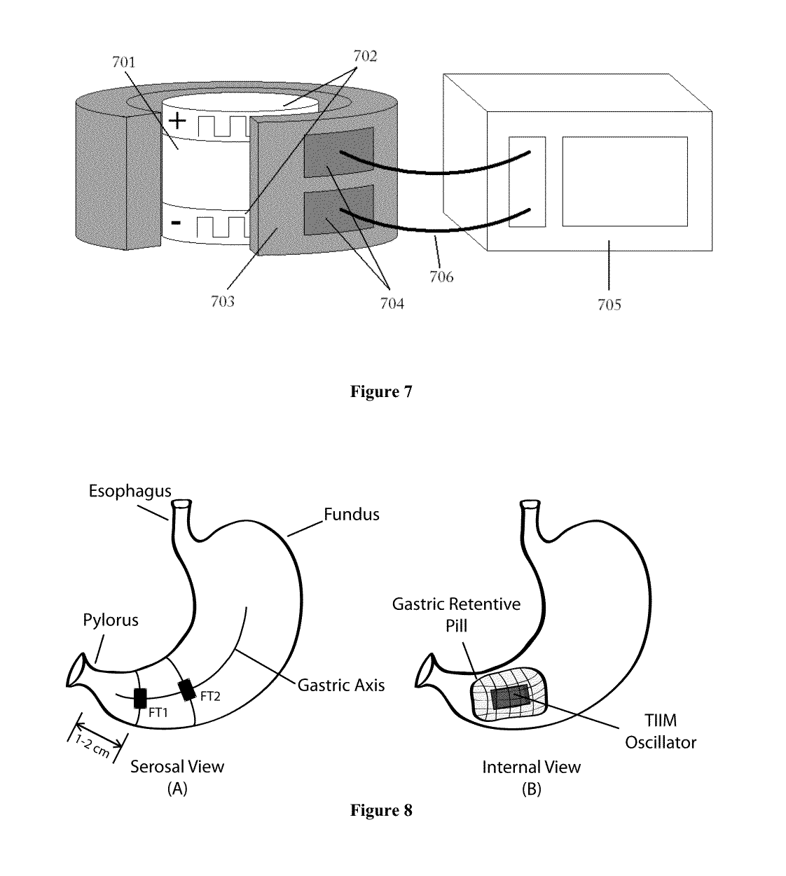

Transluminal Transverse Impedance Measurements: FIG. 7 is a schematic illustration of transluminal transverse impedance measurement. The wave source is contained within a capsule body 701 as described and shown in FIG. 5. Interrogating transducers 702 can be allowed to come into contact with the lumen of the gastrointestinal organ 703 chosen for proximity to the organ of interest. Cutaneous sensors (signal detector) 704 detect the induced phenomena from (or the signal generated by) the interrogating transducer 702. In one particular embodiment, the signal detector detects the signal emitted by the interrogating transducer 702 but is attenuated by the tissue 703 of the organ of interest. The measurement of the physical phenomenon correlating to the interrogating signal is converted to a low power electrical signal by the cutaneous sensors, which can then be transmitted via wires 706 or any other suitable medium to the final block 705, which can optionally encompass the signal conditioning, A/D conversion, processing, memory, and/or display unit shown and described in FIG. 1.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Materials and Methods: TIIM Capsule Design: For the present study the TIIM transducer was implemented as a gastric retentive pill, although it could also be positioned on the tip of a transnasal or transoral catheter. Each custom-designed TIIM transducer contained a miniature, battery-supplied, 50 kHz, 1.5 V oscillator (Linear Technology, Milpitas, Calif., USA). The length of the transducer was 18 mm with a diameter of 11 mm. It was embedded in dry, biocompatible super-absorbent polymer granules contained in a nonwoven permeable polyvinyl alcohol mesh (20-gsm) inside a size AAA DB capsule (Capsugel, Morristown, N.J., USA). The polymer granules swelled to 30-40 times their dry size in gastric liquid. The parameters of the expandable capsule were chosen to have minimal impact on the stomach and to maintain ease of swallowing, while still being able to ensure gastric retention, even during inter-digestive periods. The permeable gastric-retentive capsule design has self-disintegration capabilities in several days, with no adverse mucosal impact or evacuation/obstruction issues.

Animals and Animal Preparation: Experiments were performed on eight mongrel dogs with a mean weight of 23.8 kg±3.3 kg, four of which were administered an active TIIM capsule, while the rest were given a deactivated (battery-removed) capsule. After 24 h fasting and 12-h water deprivation, each animal ingested transorally a single capsule as described above (TIIM or sham) with 500 cc of room-temperature water. The pill swelled to its maximum size in the stomach within 15 minutes after ingestion to dimensions exceeding 1.5 cm in any direction, and subsequently was unable to pass the pyloric sphincter. The animals then underwent induction with an intravenous injection of thiopental (Thiotal 15 mg kg⁻¹ IV, Vetoquinol Canada, Lavaltrie, QC, Canada) and were subsequently maintained on inhalant isoflurane and oxygen (Halocarbon Laboratories, River Edge, N.J., USA) with a vaporizer setting of 1%-3%. The anesthesia was chosen because it did not influence gastric neurotransmitters, and as such would not affect gastric contractions. Individually the animals were then positioned supine, their abdomens shaved, cleaned, and sterilized with alcohol before performing laparotomy via a median incision vertically along the linea alba to gain access to the stomach.

After the incision, the position of the ingested pill was verified endoscopically using an EPK-700 veterinary endoscope (Pentax, Tokyo, Japan), and the serosa of the stomach was measured using an oscilloscope (Tektronix, Beaverton, Ore., USA) to confirm the presence of an activated or deactivated pill. After this verification, two 90W24 force transducers (RB Products, Stillwater, Minn., USA), specifically designed for gastric motility monitoring were surgically sutured to the serosal side of the antral stomach along the gastric axis. The first force transducer (FT1) was positioned 1-2 cm from the pylorus, and the second (FT2) was affixed proximally 5-6 cm from the pylorus (FIG. 8A). The intraluminal position of the gastric-retentive pill is shown in FIG. 8B.

The signals from the force transducers were amplified using a custom-designed multichannel bridge amplifier, and digitized using a PCMCIA DAQ Card-AL-16XE-50 (National Instruments, Austin, Tex., USA). The FT signals were monitored and analyzed with custom-designed signal processing and visualization software (GAS-6.2, Biomedical Instrumentation Laboratory, University of Calgary, Calgary, Alberta, Canada). Once the force transducers were in place, and before contractions were induced, their functionality was verified mechanically and the offsets and gains were calibrated accordingly.

Following the FT implantations the abdomen was closed, and after appropriate skin cleaning and preparation, three pediatric ECG electrodes (Conmed, Utica, N.Y., USA) were placed cutaneously over the stomach along the abdominal projection of the gastric axis, with a ground electrode positioned closer to the left hip of the animal. The position of the electrodes was similar to the one associated with impedance epigastrography, since previous studies have suggested optimal electrode placement. See, for example, Kee et al., Annals of Biomedical Engineering 1996; 24 328-32.

The cutaneous electrodes were connected to a custom-designed multichannel electrogastrograph (EGG, James Long Company, Caroga Lake, N.Y., USA), which measured the surface electrical activity relative to ground. The cut-off frequency of the low-pass filter of the EGG amplifier was set to 0.1 Hz. The signals were then digitized using the same PCMCIA card DAQ Card-AL-16XE-50 (National Instruments, Austin, Tex., USA) simultaneously with the FT signals, and were subsequently monitored and stored for further analysis using the same custom software.

Experimental Procedure: After the experimental setup was completed, a baseline recording was performed with no pharmacological stimulant for 30 minutes. Following this recording, neostigmine (0.04 mg kg⁻¹, APP Pharmaceuticals, Schaumburg, Ill.) was administered intravenously as a smooth muscle stimulant to invoke contractions. Thirty minutes of invoked contractile activity were subsequently recorded. The total recorded time from each animal was one hour; ½ hour basal state, and ½ hour invoked contractile state, with a one-minute time interval between them for the administration of neostigmine.

At the end of the experiments the animals were sacrificed by an intravenous injection of Euthanyl, 480 mg/4.5 kg (Bimeda-MTC Animal Health Inc., Cambridge, ON, Canada). Subsequent retrieval of the expanded pill was performed in order to verify its retention within the stomach, and to confirm the presence of signal in the active TIIM pills or the lack thereof in the inactive sham pills using an oscilloscope. The post-administration volume of each gastric-retentive pill was measured to quantify expansion dimensions.

Signal Processing and Statistics: Since the cutaneous and the force transducer measurements were relativistic in nature, all measurements (2 cutaneous and 2 FT channels) were normalized with the maximal amplitude becoming unity and the minimal set to zero. Thirty one-minute gastric motility indices were calculated for each channel per test (basal or post-neostigmine) from the normalized measurements, and the corresponding values (TIIM VS FT) were comparatively assessed using the Pearson Correlation coefficient (PCC). For each 30-sample correlation a PCC of 0.349 or greater was considered to be statistically significant (p<0.05).

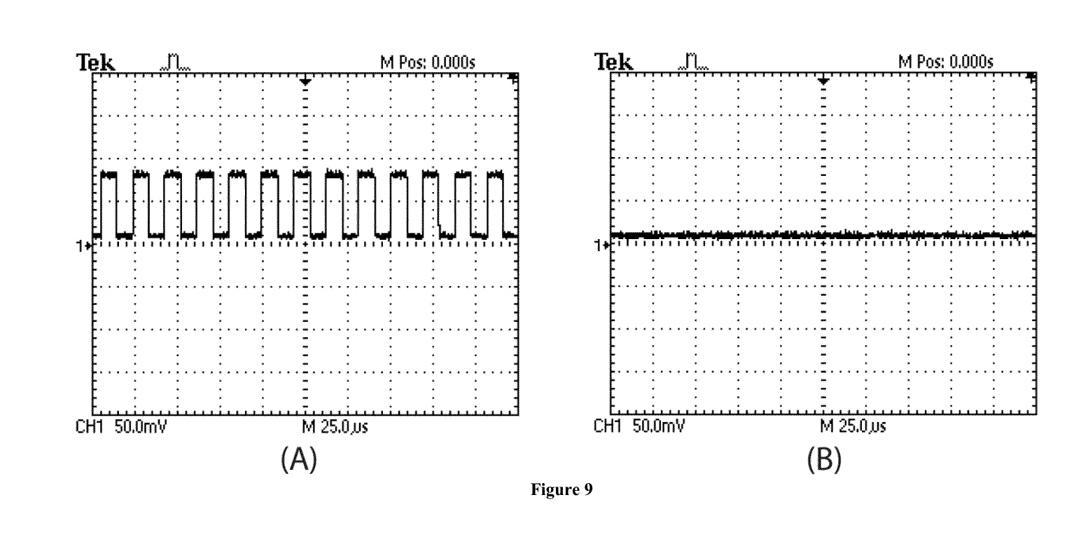

Results: Prior to the force transducer implantation, the electrical activity or lack thereof of the ingested pill was verified with an oscilloscope. FIG. 9A shows a sample tracing of an active pill in the stomach measured from the serosa, while FIG. 9B depicts a sample tracing of an inactive sham pill. Final verification of the pill's activity at the conclusion of each experiment revealed that all TIIM pills had remained active for the entire duration of the test, and conversely, sham pills remained inactive as anticipated.

In each animal the expanded pill was retrieved at the end of the experiment, and was confirmed to have a volume of 12±0.4 ml, with dimensions exceeding 1.5 cm in all directions. In each case the pill was removed from the stomach, indicating that the contractions had not been able to propel the expanded gastric-retentive capsule beyond the pyloric sphincter.

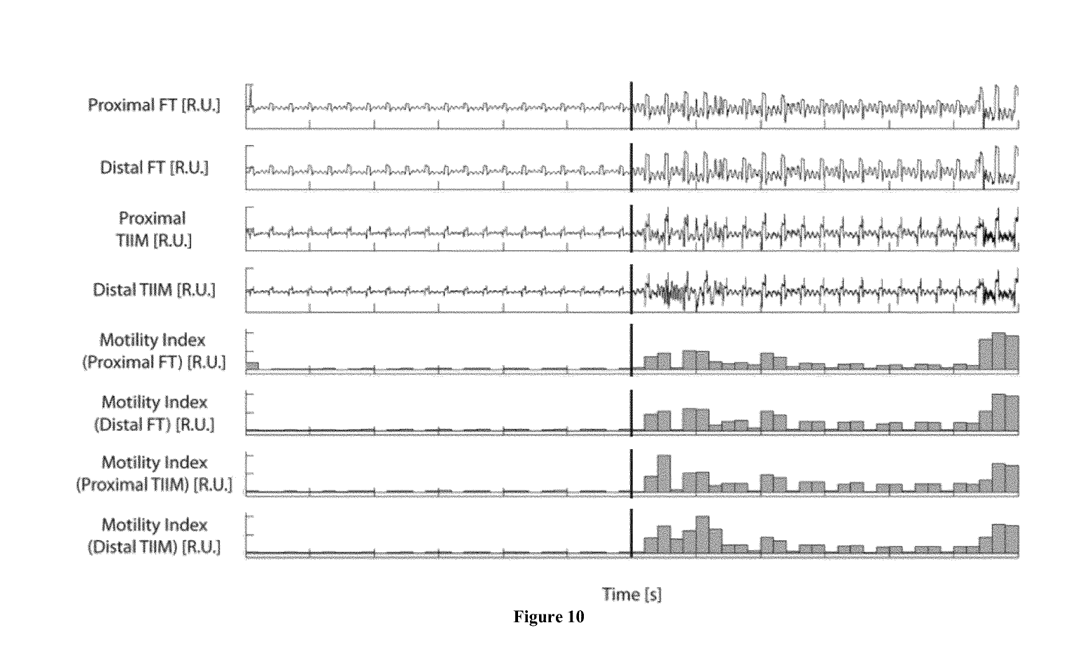

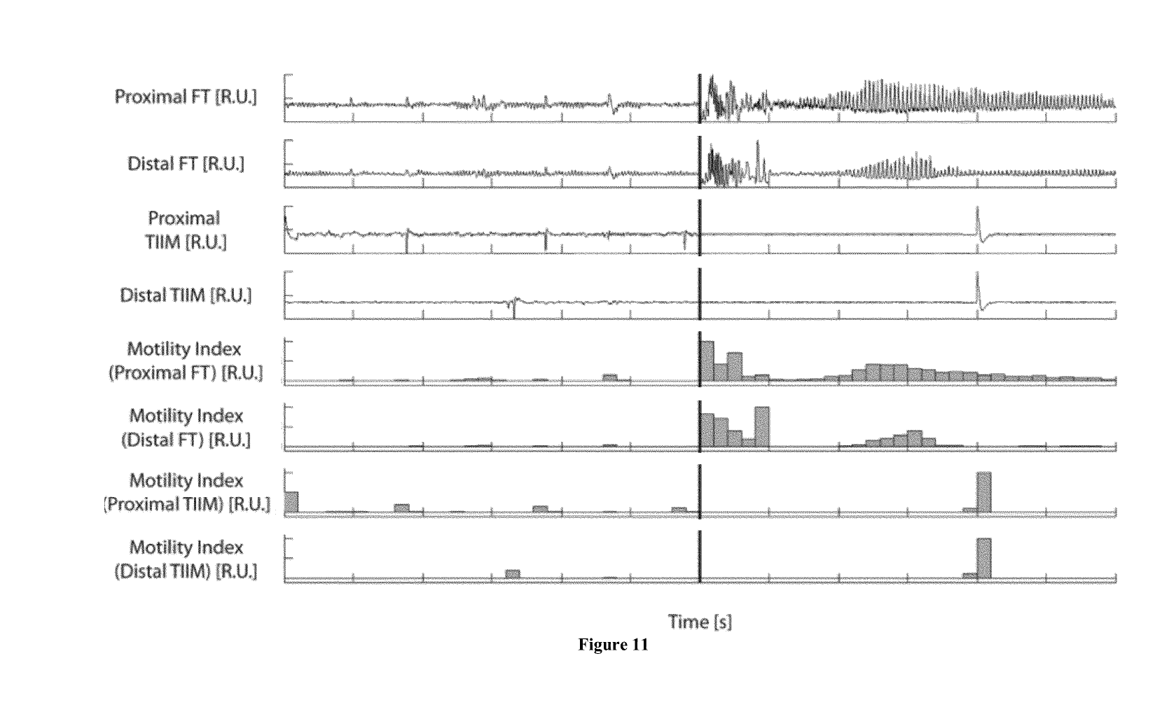

A typical example of simultaneous FT and TIIM recordings for an activated pill, as well as their one-minute motility indices is shown in FIG. 10. The combined plots present 30 minutes of basal activity, followed by 30 minutes of pharmacologically-induced contractions. During the baseline test there was varying evidence of spontaneous contractile activity. This was often more constant and exhibited less variability than in the test involving neostigmine-induced contractions. In both cases (basal or induced contractions) there were statistically-significant (p<0.05) correlations between the TIIM motility indices and the FT motility indices. In the case of the deactivated sham pill (FIG. 11) the results showed no statistically significant correlations between the respective motility indices. Table 1 lists the Pearson correlation coefficients of the calculated motility indices, with an asterisk denoting statistical significance (p<0.05).

TABLE 1
Summary comparison of the Pearson Correlation
Coefficients of the motility indices.

| | | Baseline | | Neostigmine | |
|---|---|---|---|---|---|
| | Dog | Proximal | Distal | Proximal | Distal |
| FT-TIIM | 1 | 0.362* | 0.856* | 0.633* | 0.560* |
| | 2 | 0.970* | 0.986* | 0.796* | 0.804* |
| | 3 | 0.853* | 0.880* | 0.868* | 0.889* |
| | 4 | 0.850* | 0.603* | 0.626* | 0.681* |
| FT-Sham | 5 | 0.165 | 0.106 | 0.116 | 0.093 |
| | 6 | 0.181 | 0.066 | 0.027 | 0.018 |
| | 7 | 0.112 | 0.054 | 0.063 | 0.061 |
| | 8 | 0.183 | 0.058 | 0.247 | 0.032 |

Asterisk indicates statistical significance (p < 0.05).
All TIIM motility indices showed generally excellent correlation with gastric contractions and were highly statistically significant.
There was no correlation of the sham indices with gastric contractions.
FT—Force Transducer,
TIIM—Transcutaneous Intraluminal Impedance Measurements

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A method for monitoring function of an internal organ of an animal comprising:
placing a signal emitting device within an interior of an animal's body;
placing a signal detector on the exterior surface of the animal's body and
transcutaneously measuring signal emitted by said signal emitting device; and
determining the function of an internal organ using the measured signal.

2. The method of claim 1, wherein said signal is an electrical signal.

3. The method of claim 2, wherein said signal detector comprises at least one electrode that is placed cutaneously on the animal.

4. The method of claim 1, wherein said step of transcutaneously measuring signal comprises obtaining analog signal generated by said signal emitting device.

5. The method of claim 4 further comprising the step of converting the analog signal to a digital signal.

6. The method of claim 1, wherein said step of determining the function of the internal organ using the measured signal comprises comparing the measured signal to a control signal measurement.

7. The method of claim 6, wherein said control signal measurement comprises measured signal of the animal under normal condition.

8. The method of claim 6, wherein said control signal measurement comprises measured signal of a control animal whose internal organ functions normally.

9. An apparatus for transcutaneously monitoring function of an internal organ of an animal, said apparatus comprising:
a signal generator configured to be placed within the interior of the body of an animal;
a signal detector configured to transcutaneously detect the signal generated by said signal generator, and wherein said signal detector is configured to be placed on the exterior body surface of said animal; and
an analyzer operatively connected to said signal detector, wherein said analyzer is configured to analyze the signal detected by said signal detector.

10. The apparatus of claim 9, wherein said signal generator further comprises a retaining element configured to retain said signal generator within the lumen of the internal organ of the animal for a period of time.

11. The apparatus of claim 10, wherein said retaining element comprises a biocompatible polymeric material.

12. The apparatus of claim 11, wherein said biocompatible polymeric material comprises biodegradable polymer.

13. The apparatus of claim 10, wherein said retaining element comprises an endoscope.

14. The apparatus of claim 9, wherein said signal detector is configured to generates a distinct signal based on the level of detected signal.

15. The apparatus of claim 9, wherein said signal generator generates an electrical signal.

16. The apparatus of claim 15, wherein said signal detector comprises at least one electrode.

17. The apparatus of claim 9, wherein said signal detector is configured to be placed cutaneously on the exterior of said internal organ to be monitored.
